(19) United States
(12) Patent Application Publication
(10) Pub. No.: US 2003/0049712 A1
(43) Pub. Date: Mar. 13, 2003

(54) Method of detecting protease activity in a cell

(76) Inventor: Michael Haugwitz, Mountain View, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/209,316

(22) Filed: Jul. 30, 2002

Related U.S. Application Data
(60) Provisional application No. 60/309,312, filed on Jul. 31, 2001.

(51) Int. Cl. G01N 33/567; C12Q 1/37

(52) U.S. Cl. 435/23; 435/7.21

(57) ABSTRACT

Methods and compositions for detecting the presence of an active protease in a cell are provided. A feature of the subject methods is that a protease detection fusion protein is employed to detect the protease activity of interest. The protease detection fusion protein includes first and second subcellular localization domains separated by a protease cleavage domain, where the first subcellular localization domain is dominant over the second. The protease detection fusion proteins employed in the subject methods are further characterized by having a label domain located between the protease cleavage and second subcellular localization domains. In practicing the subject methods, the protease detection fusion protein is first provided inside the cell to be assayed. Following a suitable incubation period, the subcellular location of the label domain is determined, where the location is indicative of whether or not the protease activity of interest is present in the cell. Also provided are systems and kits for use in practicing the subject methods. The subject invention finds use in a variety of different applications, including protease activity detection applications, drug screening applications, etc.

### CROSS-REFERENCE TO RELATED APPLICATIONS

Pursuant to 35 U.S.C. § 119 (e), this application claims priority to the filing date of U.S. Provisional Patent Application Serial No. 60/309,312 filed Jul. 31, 2001; the disclosure of which is herein incorporated by reference.

### INTRODUCTION

1. Field of the Invention

The field of this invention is proteases, and specifically assays therefore.

2. Background of the Invention

Assays for the presence in a cell of a protease activity typically involve lysing a, population of cells, and assaying the lysate for the presence of the protease. These assays do not allow detection of active protease in an individual cell. Thus, enzyme activity measured in such assays can be due to a very high level of activity in a small number of cells, or a low level of activity in a large number of cells, but these possibilities cannot be distinguished. Furthermore, since currently available methods involve assaying a cell lysate, the cells are killed, and cannot be used in further studies.

Detection of protease activity in live, individual cells is of interest in many applications, such as monitoring apoptotic events, determining the effect of a particular factor on expression of a protease-encoding gene, and determining the effect of an agent on protease activity. In particular, it is of interest in many applications that the cells remain alive, so that they can be used in further studies. Thus, there is a need in the art for methods of detecting the presence in individual cells of active protease. The present invention addresses this need.

### SUMMARY OF THE INVENTION

Methods and compositions for detecting the presence of an active protease in a cell are provided. A feature of the subject methods is that a protease detection fusion protein is employed to detect the protease activity of interest. The protease detection fusion protein includes first and second subcellular localization domains separated by a protease cleavage domain, where the first subcellular localization domain is dominant over the second. The protease detection fusion proteins employed in the subject methods are further characterized by having a label domain located between the protease cleavage and second subcellular localization domains. In practicing the subject methods, the protease detection fusion protein is first provided inside the cell to be assayed: Following a suitable incubation period, the subcellular location of the label domain is determined, where the location is indicative of whether or not the protease activity of interest is present in the cell. Also provided are systems and kits for use in practicing the subject methods. The subject invention finds use in a variety of different applications, including protease activity detection applications, drug screening applications, etc.

### BRIEF DESCRIPTION OF THE DRAWINGS

FIGS. 1 & 2 depict schematically assay methods of the invention.

### DEFINITIONS

The terms “polynucleotide” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art. The term “polynucleotide” includes double-stranded DNA found, inter alia, in linear DNA molecules (e.g., restriction fragments), viruses, plasmids, and chromosomes.

A DNA “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence may be located 3′ to the coding sequence.

The terms “DNA regulatory sequences”, and “regulatory elements”, used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding regions responsible for the binding of RNA polymerase. Eukaryotic promoters will often, but not always, contain “TATA” boxes and “CAT” boxes. Various promoters, including inducible promoters, may be used to drive expression.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

The amino acids described herein are preferred to be in the “L” isomeric form. The amino acid sequences are given in one-letter code (A: alanine; C: cysteine; D: aspartic acid; E: glutamic acid; F: phenylalanine; G: glycine; H: histidine; I: isoleucine; K: lysine; L: leucine; M: methionine; N: asparagine; P: proline; Q: glutamine; R: arginine; S: serine; T: threonine; V: valine; W: tryptophan; Y: tyrosine; X: any residue). NH₂ refers to the free amino group present at the amino terminus of a polypeptide. COOH refers to the free carboxyl group present at the carboxyl terminus of a polypeptide. In keeping with standard polypeptide nomenclature, J Biol. Chem., 243 (1969), 3552-59 is used.

A “host cell”, as used herein, denotes microorganisms or eukaryotic cells or cell lines cultured as unicellular entities which can be, or have been, used as recipients for recombinant vectors or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

A recombinant vector (also referred to herein as a “construct”) is “introduced” into a cell, e.g., an isolated cell (e.g., a cell in in vitro culture), i.e., a construct is made to enter the cell using any known method, including, but not limited to, transformation, transfection, electroporation, calcium phosphate precipitation, microinjection, infection, and the like.

### DESCRIPTION OF THE SPECIFIC EMBODIMENTS

Methods and compositions for detecting the presence of an active protease in a cell are provided. A feature of the subject methods is that a protease detection fusion protein is employed to detect the protease activity of interest. The protease detection fusion protein includes first and second subcellular localization domains separated by a protease cleavage domain, where the first subcellular localization domain is dominant over the second. The protease detection fusion proteins employed in the subject methods are further characterized by having a label domain located between the protease cleavage and second subcellular localization domains. In practicing the subject methods, the protease detection fusion protein is first provided inside the cell to be assayed. Following a suitable incubation period, the subcellular location of the label domain is determined, where the location is indicative of whether or not the protease activity of interest is present in the cell. Also provided are systems and kits for use in practicing the subject methods. The subject invention finds use in a variety of different applications, including protease activity detection applications, drug screening applications, etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a protease” includes a plurality of such proteases and reference to “the fluorescent protein” includes reference to one or more proteins and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the methods for detecting protease activity, as well as fusion protein compositions employed therein, are described first in greater detail, followed by a review of representative applications in which the subject methods and compositions find use, as well as a review of systems and kits that find use in practicing the subject methods.

Methods and Compositions for Detecting an Active Protease in a Cell

As summarized above, the subject invention provides methods and compositions for detecting the presence of an active protease in a cell, e.g., a eukaryotic cell. The methods generally involve providing a protease, detection fusion protein in the cytosol of cell to be assayed and then, following a suitable incubation period, determining the subcellular location of a label domain of the protease detection fusion protein, where the subcellular location of the label domain is indicative of whether or not the protease activity of interest is present in the cell. In further describing the subject methods, the protease detection fusion proteins employed in the subject methods are described first, followed by a more in-depth review of how the detection fusion proteins are employed in the subject methods.

Protease Detection Fusion proteins

The protease detection fusion proteins employed in the subject methods are proteins that include first and second subcellular localization domains, where the first subcellular localization domain is dominant over the second subcellular localization domain.

The first and second subcellular localization domains are domains that direct the movement of the protein to a particular subcellular location, where subcellular locations of interest include, but are not limited to: the nucleus, the cytosol, the plasma membrane, cellular organelles, e.g., mitochondria, endoplasmic reticulum (e.g., rough, smooth), golgi apparatus, etc.

By “dominant” is meant that when the first and second subcellular localization domains are present on the same fusion protein, the fusion protein is directed to the subcellular location that is the target of the first subcellular localization domain. In other words, when both localization domains are present in the same fusion protein, the first subcellular localization domain controls the location to which the fusion protein migrates, i.e., the fusion proteins migrates to the location that is the target of the first subcellular localization domain.

The subject protease-detection fusion proteins are further characterized in that the first and second subcellular localization domains are separated by a protease cleavage domain. In addition, located between the protease cleavage domain and the second subcellular localization domain is a label domain, such that the label domain is always joined to the second subcellular localization domain, whether or not the fusion protein is cleaved by a protease such that the first and second subcellular localization domains are separated from each other.

As such, the subject fusion proteins include first and second localization domains, a protease cleavage domain and a label domain. Each of these components of the subject protease detection fusion proteins is now described separately in greater detail.

First Subcellular Localization Domain

As indicated above, the first subcellular localization domain is a domain that directs a protein, i.e., targets a protein, to a first subcellular location, where subcellular locations of interest include, but are not limited to: the nucleus, the cytosol, the plasma membrane, cellular organelles, e.g., mitochondria, endoplasmic reticulum (e.g., rough, smooth), golgi apparatus, etc. A feature of the first subcellular localization domain is that it is dominant over the second subcellular localization domain, such that its activity controls the fusion protein when the fusion protein includes both the first and second subcellular localization domains.

In certain embodiments, the first subcellular localization domain is a nuclear export signal. Nuclear export signals are generally leucine-rich stretches of amino acids of from about 10 to about 100 amino acids in length that direct export of a protein from the nucleus into the cytoplasm. A variety of NES have been reported and can be used in the fusion protein in the subject methods. See, e.g., Ohno et al. (1998) Cell 92:327-336; Henderson and Eleftheriou (2000) Experimental Cell Research 256:213-224; and Huang et al. (1993) Mol. Cell Biol. 13:7476. Examples of NES include leucine-rich amino acid peptide sequences as described in CRMI protein and various viral proteins such as HIV-1 Rev protein, and EIB and E4 proteins (Ossareh-Nazari, B. et al. (1997) Science 278:141-4; Wolff, B. (1997) Chemistry and Biology 4:139-47; Dobelstein, M. (1997) EMBO J. 16(4):4276-84); Fischer et al. (1995) Cell 82:475-483. As one non-limiting example, a MAP kinase kinase NES is used, having the amino acid sequence (SEQ ID NO: 01).

The first subcellular localization domains may include a single copy of a particular localization sequence, or two or more copies of a given localization sequence, or two or more copies of different localizations sequences that nonetheless work together to provide dominance of the second subcellular localization domain. For example, a fusion protein for use in the subject methods may include one NES, and in some embodiments include more than one NES, e.g., two or more NES in tandem.

Second Subcellular Localization Domain

As indicated above, the second subcellular localization domain is a domain that directs a protein, i.e., targets a protein, to a second subcellular location, where subcellular locations of interest include, but are not limited to: the nucleus, the cytosol, the plasma membrane, cellular organelles, e.g., mitochondria, endoplasmic reticulum (e.g., rough, smooth), golgi apparatus, etc. A feature of the second subcellular localization domain is that it is dominated by the first subcellular localization domain, such that its activity does not control the fusion protein when the fusion protein includes both the first and second subcellular localization domains.

In certain embodiments, the second subcellular localization domain is a nuclear localization signal (NLS). NLSs of interest include, but are not limited to: PKKKRKV (SEQ ID NO: 02) and KKKRKVC (SEQ ID NO: 3) (Kalderon et al. (1984) Cell 39:499); GKKRSKA (SEQ ID NO: 04) (Moreland et al. (1987) Mol. Cell. Biol. 7:4048); KRPRP (SEQ ID NO: 05) (Lyons et al. (1987) Mol. Cell. Biol. 7:2451); GNKAKRQRST (SEQ ID NO: 06) (Gilmore et al. (1988) J. Virol. 62:703); GGAAKRVKLD (SEQ ID NO: 07) (Chelsky et al. (1989) Mol. Cell. Biol. 9:2487); SALIKKKKKMAP (SEQ ID NO: 08) (Van Etten et al. (1989) Cell 58:669); RKLKKLGN (SEQ ID NO: 09) (Guiochon-Mantel et al. (1989) Cell 57:1147); PQPKKKP (SEQ ID NO: 10) (Dang et al. (1989) J. Biol. Chem. 264:18019); ASKSRKRKL (SEQ ID NO: 11) (Chida et al. (1992) Proc. Natl. Acad. Sci. USA 89:4290); KKKYK (SEQ ID NO: 12) and KKKYKC (SEQ ID NO: 13), (Bukrinsky et al. (1993) Nature 365:666); KSKKK (SEQ ID NO: 14) (Bukrinsky et al. (1993), supra); and AKRVKL (SEQ ID NO: 15) and KRVKLC (SEQ ID NO: 16 (Chelsky et al. (1989), supra). Additional examples of nuclear localization signals include RRMKWKK (SEQ ID NO: 17(Moede et al. (1999) FEBS Lett. 461:229-234; and nuclear localization signals described in Boulikas (1993) Crit. Rev. Eukaryot Gene Expr. 3:193-227; Hsieh et al. (1998) J. Cell. Biochem. 70:94-109; Truant and Cullen (1999) Mol. Cell. Biol. 19:1210-1217; and Irie et al. (2000) J. Biol. Chem. 275:2647-2653.

The second subcellular localization domains may include a single copy of a particular localization sequence, or two or more copies of a given localization sequence, or two or more copies of different localization sequences that nonetheless work together to provide for targeting to the second subcelluar location, when not dominated by the first subcellular localization domain. For example, a fusion protein for use in the subject methods includes at least one NLS, and in some embodiments includes more than one NLS, e.g., two or more NLS sequences in tandem.

Protease Cleavage Sites

Separating the first and second subcellular localization domains in the subject protease detection fusion proteins is a protease cleavage site. The protease cleavage site that lies between the first and second localization domains on the subject fusion proteins is one that is cleaved by the protease of interest, i.e., the protease whose activity is to be assayed in the subject methods. Generally, the protease cleavage site is a site or domain, i.e., sequence of amino acid residues, of from about 2 to about 20, usually from about 3 to about 20 and often from about 4 or 5 to about 15 amino acid residues, where the sequence is cleaved by a cytosolic protease, i.e., a protease that is active in the cytosol of a cell. In some embodiments, from 2 to about 12, or from about 4 to about 8, additional amino acids on the carboxyl and/or amino terminus of the protease cleavage site are included, which additional amino acids are found in a native substrate of the protease.

Cytosolic proteases of interest include, but are not limited to: Caspases; Viral proteases; Bacterial toxins; Miscellaneous cytosolic proteases; “artificial” proteases; etc.

Caspases belong to a class of cysteine proteases that comprise a multi gene family with more than 12 distinct mammalian family members. Caspases play a key role during embryonal development, inflammation and cell death (For review see: Cell Death and Differentiation 1999, Vol 6, 11). The substrates cleaved by specific members of the Caspase family account for the majority of morphological changes/events that occur during cell death. A link between deregulation of apoptosis and disease in humans has been clearly established. Insufficient apoptosis can result in cancer and lymphoproliferative disorders. On the other hand it has been shown that excessive cell death has been genetically linked to muscular atrophy, and is believed to be a contributing factor in neurodegenerative disorder, trauma and stroke. Therefore, Caspases are prime drug targets if it comes to cure different diseases mentioned above. One specific Caspase of interest is Caspase 3. Caspase 3 is one of the key players in the Caspase cascade, initiated during apoptosis. Caspase 3 is called the “executer” Caspase, due to its' high activity and wider range of cellular substrates (Nicholson et al, 1995, Nature 376; 37-43; Tewari et al., 1995, Cell 81; 801-809). It has been shown, that the specific inhibition of Caspase 3 activity after a stroke can decrease the extent of secondary loss of tissue surrounding the immediately damaged tissue. However all members of the Caspase family are potential drug targets due to there substrate specificity and involvement in different apoptotic pathways. Other specific Caspase family members of interest include, but are not limited to: Caspases of particular interest include: caspase 2, caspase 6, caspase 8 and caspase 9, etc.

Another class of cytosolic proteases of interest, i.e., proteases that may be present and active in the cytosol, are retroviral proteases. Proteolytic processing at specific sites in the Gag and Gag-Pro-Pol precursor by a viral encoded protease is an essential step in the viral life cycle. Since the protease has a central role in proteolytic processing, it provides an important target for the design of inhibitors of viral replication. Normally the viral protease is expressed as an inactive form activated in the fully assembled and already budded virus particle (Witte and Baltimore 1978; J. Virol., 26; 750-761). However a premature activation of the viral protease in the cytosol has been found during HIV-1 infection (Kaplan and Swanstrom, 1991, Proc. Natl. Acad. Sci.,88; 4528-4532). In addition, overexpression of the Gag-Pro-Pol precursor induces premature activation of the protease in the cytosol as well (Karacostas et al., 1993, Virology 145; 280-292) The presence of processed viral proteins in infected cells demonstrates the presence of viral protease activity in the cytosol. As such, the viral protease may also cleave cellular proteins. Therefore, the subject methods can be used to monitor viral protease activity in the cytosol of infected cells by modifying the invention so that it contains an amino acid sequence, specifically recognized and cleaved by the viral protease. As such, in certain embodiments, viral protease cleavage sites are of interest in the subject protease detection fusion proteins.

Yet another type of proteases that are of interest are bacterial toxin proteases. Specific bacterial toxins, like the tetanus or botulinum toxin, exhibit protease activity, i.e., have a proteolytic activity. The presence of those toxins in the cytosol of mammalian cells causes the cleavage of proteins on secretory/synaptic vesicles essential for the fusion of those vesicles with the plasmamembrane. By inhibiting the fusion of the vesicles with the plasmamebrane, the content of those vesicles, mainly neurotransmitter, will not be released into the extracellular space of neuro-muscular junctions, causing the loss of communication between the neuronal network and muscles. Therefore bacterial toxins with a cytosolic protease activity can be a seen as a drug target in the effort to find drugs to inhibit the toxic effect of bacterial proteases in the cytosol. As such, in certain embodiments, the protease cleavage site is a bacterial toxin protease cleavage site.

Additional cytosolic protease cleavage sites of interest include, but are not limited to: Aminopeptidases, such as Cytosol aminopeptidase (Leucyl Aminopeptidase, e.g., Cathepsin III; Dipeptidase, such as Cytosol non-specific dipeptidase (DPPII) and Cystein glycin-S-conjugate dipeptidase; Cytosol alanyl aminopeptidase; Calpain; etc.

Yet another class of proteases of interest are “Artificial Proteases.” Artificial proteases are defined as chimeric and/or truncated proteases. These proteases do not exist endogenously in the cytosol, but are engineered proteins (either fusion and/or truncated proteins) that contain a specific active protease domain and are targeted to the cytosol. The activity of such an artificial protease in the cytosol can be monitored by the invention if the invention contains the specific cleavage sequence recognized by the protease domain of the artificial protease. Representative protease domains of interest include, but are not limited to: extracellular or secreted proteases, e.g., matrix metalloproteases, serine proteases, etc. In these embodiments, the protease domain of the subject fusion proteins may be recognized by any protease, including secreted proteases, such as the specific secreted proteases mentioned above.

Specific proteolytic cleavage sites are known to those skilled in the art; a wide variety are known and have been described amply in the literature, including, e.g., Handbook of Proteolytic Enzymes (1998) A J Barrett, N D Rawlings, and J F Woessner, eds., Academic Press. Proteolytic cleavage sites include, but are not limited to, an enterokinase cleavage site: (Asp)₄Lys (SEQ ID NO: 18 a factor Xa cleavage site: Ile-Glu-Gly-Arg (SEQ ID NO: 19 a thrombin cleavage site, e.g., Leu-Val-Pro-Arg-Gly-Ser (SEQ ID NO: 20 a renin cleavage site, e.g., His-Pro-Phe-His-Leu-Val-Ile-His (SEQ ID NO: 21 a collagenase cleavage site, e.g., X-Gly-Pro (where X is any amino acid); a trypsin cleavage site, e.g., Arg-Lys; a viral protease cleavage site, such as a viral 2A or 3C protease cleavage site, including, but not limited to, a protease 2A cleavage site from a picornavirus (see, e.g., Sommergruber et al. (1994) Virol. 198:741-745), a Hepatitis A virus 3C cleavage site (see, e.g., Schultheiss et al. (1995) J. Virol. 69:1727-1733), human rhinovirus 2A protease cleavage site (see, e.g., Wang et al. (1997) Biochem. Biophys. Res. Comm. 235:562-566), a picornavirus 3 protease cleavage site (see, e.g., Walker et al. (1994) Biotechnol. 12:601-605; and a caspase protease cleavage site, e.g., DEVD (SEQ ID NO: /) recognized and cleaved by activated caspase-3, where cleavage occurs after the second aspartic acid residue.

Label Domain

In addition to the first and second subcellular localization domains and the protease cleavage domains, as described above, the subject protease detection fusion proteins also include a label domain. The label domain is located in the fusion protein such that upon cleavage of the fusion protein, it remains bound to the second subcellular localization domain. As such, the label domain is positioned between the protease cleavage site and the second subcellular localization domain.

The label domain of the subject protease detection fusion proteins is either directly or indirectly detectable. Examples of directly detectable domains are domains that are, by themselves, directly detectable, such as fluorescent proteins, etc. Examples of indirectly detectable domains are domains that are detectable when visualized with one or more additional components of a signal producing system. An example of an indirectly detectable label domain is a domain or epitope that is recognized by an antibody, where when the antibody is present with the fusion protein it binds to the fusion protein to provide for a detectable fusion protein. The detecting antibody may itself be directly or indirectly detectable. Examples of directly detectable antibodies are fluorescently labeled antibodies, isotopically labeled antibodies, etc. Examples of indirectly detectable antibodies are antibodies that are detected by a directly labeled secondary antibody, antibodies that include an enzymatic moiety that converts a substrate to a directly detectable, e.g., chromogenic product, etc.

In many embodiments, the label domain is a fluorescent protein. As used herein, the term “fluorescent protein” refers to any protein capable of fluorescence when excited with appropriate electromagnetic radiation. This includes fluorescent proteins whose amino acid sequences are either naturally occurring or engineered (i.e., mutants or analogs). Fluorescent proteins of interest include, but are not limited to: (1) the Aequoria victoria green fluorescent proteins and variants thereof, such as those described in U.S. Pat. Nos.: 6,066,476; 6,020,192; 5,985,577; 5,976,796; 5,968,750; 5,968,738; 5,958,713; 5,919,445; 5,874,304; and 5,491,084; the disclosures of which are herein incorporated by reference, as well as International Patent Publications: WO 00/46233; WO 99/49019; and DE 197 18 640; and the Anthozoa derived fluorescent proteins, including but no limited to: (1) amFP485, cFP484, zFP506, zFP540, drFP585, dsFP484, asFP600, dgFP512, dmFP592, as disclosed in application Ser. No. 10/006,922, the disclosure of which is herein incorporated by reference; (2) hcFP640, as disclosed in application Ser. No. 09/976,673, the disclosure of which is herein incorporated by reference; (3) CgCP, as disclosed in application serial No. 60/255,533, the dislcosure of which is herein incorporated by reference; and (4) hcriGFP, zoanRFP, scubGFP1, scubGFP2, rfloRFP, rfloGFP, mcavRFP, mcavGFP, cgigGFP, afraGFP, rfloGFP2, mcavGFP2, mannFP, as disclosed in application serial No. 60/332,980, the dislcosure of which is herein incorporated by reference; etc.

Methods of Using the Protease Detection Fusion Proteins

The above described protease detection fusion proteins are used to detect the activity of a protease in a cell, where the methods of using the subject fusion proteins typically include the following steps. First, a protease detection fusion protein is provided in a cell to be assayed for protease activity. Specifically, the fusion protein is provided in the cytosol of the cell to be assayed. The fusion protein may be provided in the cytosol of the cell using any convenient protocol. As such, the fusion protein may be introduced directly into the cell using any convenient protein introduction protocol, e.g., microinjection, etc., where numerous different protocols for injecting a protein into a cell are known in the art.

Alternatively, a nucleic acid acid, e.g., vector comprising a coding sequence for the subject fusion proteins, may be introduced into the cell to be assayed, where the encoded fusion protein is expressed in the cell following introduction. Representative vectors that find use in the subject methods are described in more detail below in the section entitled Recombinant Vectors and Host Cells. In many such embodiments, the vector employed is a eukaryotic expression vectors, where representative expression vectors of interest include, but are not limited to: pSVK3, pSVL, pMSG, pCH110, pMAMneo, pMAMneo-LUC, pPUR, and the like.

Generally, the expression cassette will be a plasmid that provides for expression of the encoded subject fusion polypeptide under appropriate conditions, i.e. in the target cell to be assayed. The expression vector will typically comprise a replicon, which includes the origin of replication and its associated cis-acting control elements. Representative replicons that may be present on the expression vector include: pMB1, p15A, pSC101 and ColE1. Expression vectors generally have convenient restriction sites located near the promoter sequence to provide for the insertion of nucleic acid sequences encoding heterologous proteins.

In addition, the expression vector may also include a marker which provides for detection of the clones that have been transformed with the vector. A variety of markers are known and may be present on the vector, where such markers include those that confer antibiotic resistance, e.g. resistance to ampicillin, tetracycline, chloramphenicol, kanamycin (neomycin), markers that provide for histochemical detection, etc. Specific vectors that may find use in the subject methods include: pBR322; pUC18, pUC19, pcDNA, and the like. Introduction of the nucleic acid encoding the subject fusion protein product into the expression vector is accomplished by cutting the expression vector and inserting the polynucleotide encoding the desired product.

In these embodiments, the expression vector is introduced into the target cell to be assayed for production of the subject fusion polypeptide, i.e., the to be assayed target cell is transformed with the expression vector. Transformation of target cells in these embodiments may be accomplished in any convenient manner, where two representative means of transformation are treatment with divalent cation transformation compositions and electrotransformation. In transformation through divalent cation treatment, the host cells are typically incubated with the one or more divalent cations, e.g. CaCl₂, which serves to make the host cell permeable to the vector DNA. See Cohen et al. (1972) Proc. Nat'l. Acad. Sci. USA 69:2110. Other agents with which the host cells may also be incubated include DMSO, reducing agents, hexaminecobalt and the like, where such agents serve to improve the efficiency of transformation. In electrotransformation (also known as transformation by electroporation) target cells are subject to an electrical pulse in the presence of the vector in a manner sufficient for the vector to enter the host cells. See Dower et al. (1988) Nucleic Acids Research 16:6127. In some embodiments, the construct is stably introduced into the cell (e.g., the construct integrates into the genome of the cell or is stably maintained as an extrachromosomal element). In other embodiments, the construct is transiently maintained in the cell.

In yet other embodiments, the to be assayed cell is one that has been pre-engineered to express the protease detection fusion protein. The cell may be one that is engineered to constitutively express the fusion protein, or express the fusion protein in response to a stimulus.

The above three protocols merely provide representative approaches to providing the fusion protein in a cell to be-assayed for protease activity, and are in no way limiting.

Following the above described first step of providing the fusion protein in the cell to be assayed, the cell is maintained for a period of time sufficient for the fusion protein to be cleaved by its corresponding protease activity, if the protease activity of interest is present. A protease activity corresponds to a given fusion protein if it cleaves the protease cleavage domain of the given fusion protein, i.e., the fusion protein is designed to be cleaved by the protease to which it is corresponds. The incubation period may vary depending on the nature of the cell, the nature of the fusion protein and its corresponding protease. Typically, this incubation period is at least about 1 minute, sometimes at least about 5 minutes and more often at least about 10 minutes, where in many embodiments the incubation period is at least about 1 hour, 6 hours, 12 hours, 1 day, 2 days, etc. The incubation temperature may vary, but is typically between about 30 and about 40° C, usually between about 35 and 38° C.

Following the incubation period, as described above, the subcellular location of the label domain in the cell is then determined. The location of the subcellular domain is determined using any convenient protocol, where the protocol employed necessarily depends on the nature of the label domain of the fusion protein. For example, where the label domain is a directly detectable fluorescent protein, any convenience fluorescent protein imaging protocol may be employed, e.g., conventional fluorescent microscopy.

Once the subcellular location of the label domain is identified or determined, the information regarding the subcellular location is then employed to determine the activity or lack thereof of the protease of interest in the cell. For example, where the label domain is present in the first subcellular location following the incubation period, a determination is made that the cell lacks the protease activity of interest, because no cleavage of the fusion protein occurred and therefore all of the fusion protein ended up in the first subcellular location, as directed by the dominant first subcellular localization domain. Alternatively, where the label domain appears in the second subcellular location following incubation period, a determination is made that the cell includes the protease of interest, since the fusion protein was cleaved thereby separating the first and second localization domains from each other and translocating the label domain to the second subcellular location.

The assays described above may be qualitative or quantitative, such that one may use the above described assays to: (a) obtain a simple yes or no answer to the question of whether the protease of interest is in the cell; as well as (b) obtain an at least semi-quantitative determination of how much protease activity is present in the cell, e.g., by comparing to a control cells that do and/or do not include the protease activity of interest, by looking at the amount of signal present in the first and second locations and relating these amounts to the activity of the protease in the cell, etc.

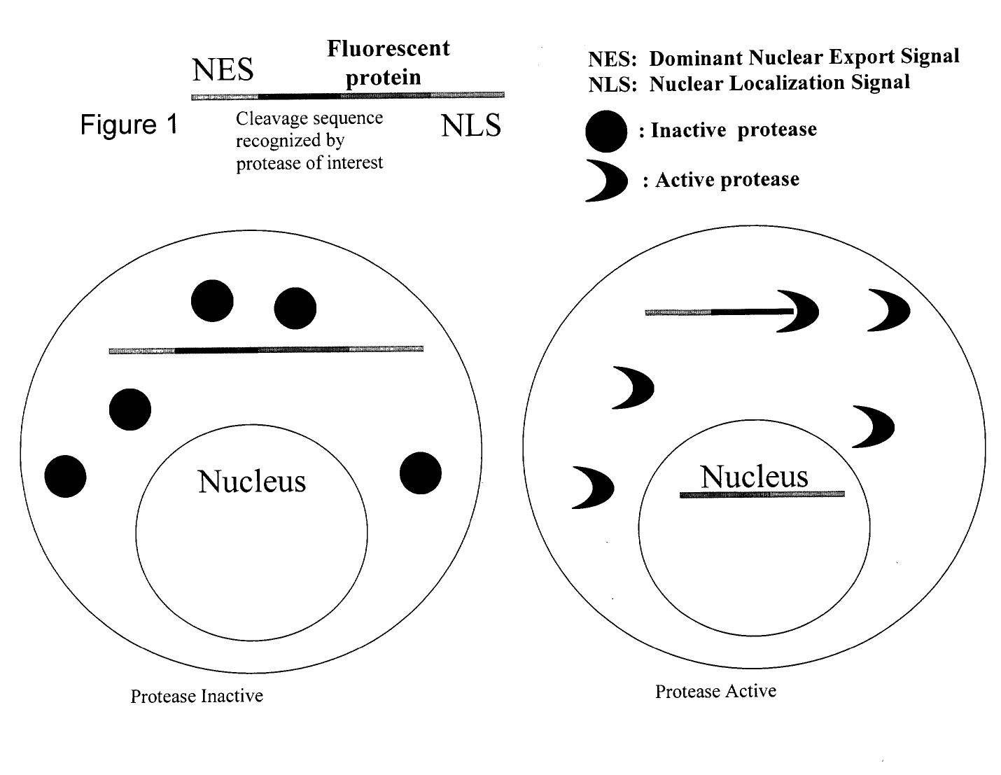

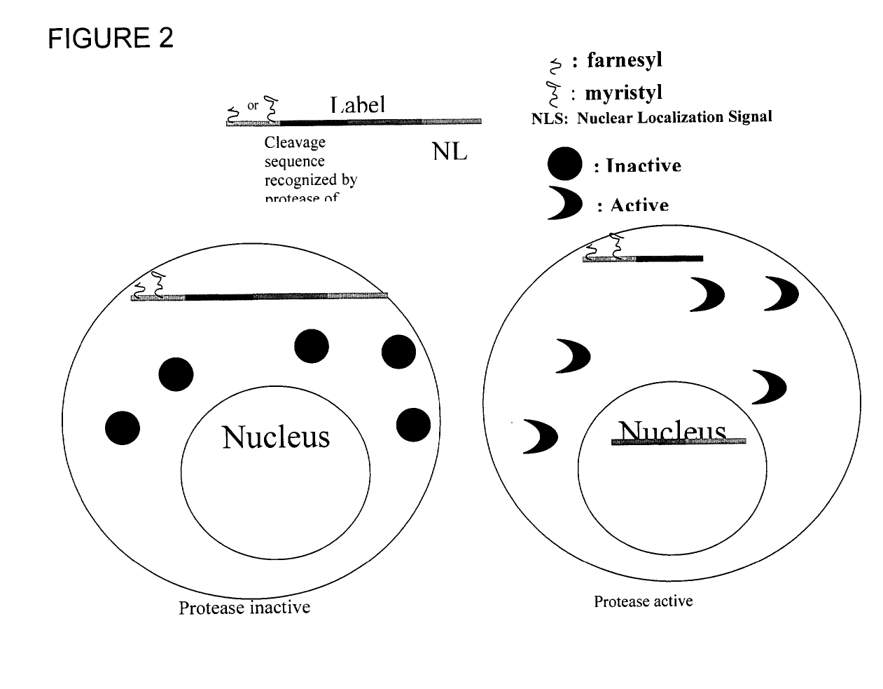

As indicated above, in some embodiments, the methods involve introducing into a eukaryotic cell a construct encoding a fusion protein which includes a nuclear export signal (NES), a label domain, e.g., a fluorescent protein, a nuclear localization signal (NLS), and a cleavage recognition site for the active protease. Translocation of the fusion protein from the cytoplasm to the nucleus (in the case of proteases located in the cytoplasm), or from the nucleus to the cytoplasm (in the case of proteases located in the nucleus), is the readout for the presence of active protease in the cell. An example of a subject method is depicted schematically in FIG. 1.

In some embodiments, the protease cleavage site is positioned adjacent to the NES such that the active protease cleaves the NES from the remainder of the fusion protein. In these embodiments, the NES is dominant over the NLS in the fusion protein and, because of this, the fusion protein remains in the cytoplasm until acted on by active protease that recognizes the protease cleavage site. Once the NES is removed by action of the active protease, the fusion protein is translocated into the nucleus.

In other embodiments, the protease cleavage site is positioned adjacent to the NLS such that the active protease cleaves the NLS from the remainder of the fusion protein. In these embodiments, the NLS is dominant over the NES in the fusion, protein and, because of this, the fusion protein remains in the nucleus until acted on by active protease in the nucleus that recognizes the protease cleavage site. Once the NLS is removed by action of the active protease, the fusion protein is translocated into the cytoplasm.

Individual cells can be analyzed for the presence of an active protease. Cells of interest include any cell having a nucleus, including, but not limited to, yeast cells; fungal cells; animal cells, including, but not limited to, frog cells (e.g., Xenopus laevis), fish cells (e.g., Zebrafish), Caenorhabditis elegans, insect cells, and mammalian cells (e.g., HEK293 cells, NIH3T3 cells, COS cells, and the like; and plant cells (e.g., Arabidopsis), including monocotyledons and dicotyledons. The subcellular location of the fluorescent protein can be determined using any known method, and is generally carried out by visual inspection of cells using a fluorescent microscope, a laser confocal microscope, and the like. Using such a visual detection system, protease activity can be detected in real time, in a living cell.

Recombinant Vectors and Host Cells

The present invention further provides recombinant vectors (“constructs”) for use in the methods of the invention, as well as recombinant host cells comprising a recombinant vector of the invention. Recombinant vectors are useful for propagation of subject polynucleotides encoding fusion proteins described herein (cloning vectors). They are also useful for effecting expression of a subject polynucleotide in a cell (expression vectors). Some vectors accomplish both cloning and expression functions. The choice of appropriate vector is well within the skill of the art. Many such vectors are available commercially.

In some embodiments, a recombinant vector includes a nucleotide sequence that encodes a fusion protein that includes a first localization signal that results in localization of the fusion protein to a first subcellular location; a label domain, e.g., a fluorescent protein; a second localization signal that results in localization of the fusion protein to a second subcellular location, such that the first localization signal is dominant over the second localization signal, such that the fusion protein is localized to the first subcellular location; and a protease cleavage site recognized by the active protease positioned between the first localization signal and the remainder of the fusion protein, such that, in the presence of the active protease, the first localization signal is cleaved from the remainder of the fusion protein.

In some embodiments, the recombinant vector includes, in order from 5′ to 3′, a transcription control sequence, a nucleotide sequence encoding an NES, a restriction endonuclease recognition site (for insertion of a sequence encoding a protease cleavage site), a nucleotide sequence encoding a fluorescent protein, and a nucleotide sequence encoding an NLS. In other embodiments, the recombinant vector comprises, in order from 5′ to 3′, a transcription control sequence, a nucleotide sequence encoding an NLS, a nucleotide sequence encoding a fluorescent protein, a restriction endonuclease recognition site (for insertion of a sequence encoding a protease cleavage site), and a nucleotide sequence encoding an NES. In many of these embodiments, the NES is dominant over the NLS.

In other embodiments, the recombinant vector comprises, in order from 5′ to 3′, a transcription control sequence, a nucleotide sequence encoding an NES, a nucleotide sequence encoding a protease cleavage site, a nucleotide sequence encoding a fluorescent protein, and a nucleotide sequence encoding an NLS. In other embodiments, the recombinant vector comprises, in order from 5′ to 3′, a transcription control sequence, a nucleotide sequence encoding an NLS, a nucleotide sequence encoding a fluorescent protein, a nucleotide sequence encoding a protease cleavage site, and a nucleotide sequence encoding an NES. In many of these embodiments, the NES is dominant over the NLS.

The recombinant vector typically further comprises a nucleotide sequence encoding a selectable marker (e.g., antibiotic resistance), and an origin of replication, e.g., for maintenance in a eukaryotic cell, or for propagation in a prokaryotic cell. For expression, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to the subject gene, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. After introduction of the DNA, the cells containing the construct may be selected by means of a selectable marker, the cells expanded and then used for expression. A variety of host-vector systems may be utilized to propagate and/or express the subject polynucleotide. Such host-vector systems represent vehicles by which coding sequences of interest may be produced and subsequently purified, and also represent cells that may, when transformed or transfected with the appropriate nucleotide coding sequences, produce fusion polypeptides of the invention. These include, but are not limited to, microorganisms (e.g., E. coli, B. subtilis) transformed with recombinant bacteriophage vectors, plasmid DNA, or cosmid DNA vectors comprising the subject polynucleotides; yeast (e.g., Saccharomyces, Pichia) transformed with recombinant yeast vectors comprising subject polynucleotides); insect cell systems (e.g., Spodoptera frugiperda) infected with recombinant virus expression vectors (e.g., baculovirus vectors, many of which are commercially available, including, for example, pBacPAK8, and BacPAK6) comprising subject polynucleotides; plant cell systems; or mammalian cell systems (e.g., COS, CHO, BHK, 293, 3T3) harboring recombinant vectors comprising mammalian promoters (e.g., metallothionein promoter) or promoters from viruses which replicate in mammalian cells (e.g., adenovirus late promoter; vaccinia virus promoter, and the like).

Examples of prokaryotic cloning vectors which find use in propagating polynucleotides of the invention are pBR322, M13 vectors, pUC18, pcDNA, and pUC19. Prokaryotic expression vectors which find use in expressing subject polypeptides in prokaryotic cells include pTrc99A, pK223-3, pEZZ18, pRIT2T, and pMC1871.

Eukaryotic expression vectors which find use in expressing subject polynucleotides and subject fusion polypeptides in eukaryotic cells include commercially available vectors such as pSVK3, PSVL, pMSG, pCH110, pMAMneo, pMAMneo-LUC, pPUR, and the like.

Generally, the expression cassette will be a plasmid that provides for expression of the encoded subject fusion polypeptide under appropriate conditions, i.e. in a host cell. The expression vector will typically comprise a replicon, which includes the origin of replication and its associated cis-acting control elements. Representative replicons that may be present on the expression vector include: pMB1, p15A, pSC101 and ColE1. Expression vectors generally have convenient restriction sites located near the promoter sequence to provide for the insertion of nucleic acid sequences encoding heterologous proteins.

In addition, the expression vector will also typically comprise a marker which provides for detection of the clones that have been transformed with the vector. A variety of markers are known and may be present on the vector, where such markers include those that confer antibiotic resistance, e.g. resistance to ampicillin, tetracycline, chloramphenicol, kanamycin (neomycin), markers that provide for histochemical detection, etc. Specific vectors that may find use in the subject methods include: pBR322, pUC18, pUC19, pcDNA, and the like. Introduction of the nucleic acid encoding the subject peptidic product into the expression vector is accomplished by cutting the expression vector and inserting the polynucleotide encoding the desired product.

Following preparation of the expression vector comprising the nucleic acid, the expression vector will be introduced into an appropriate host cell for production of the subject fusion polypeptide, i.e. a host cell will be transformed with the expression vector. Transformation of host cells may be accomplished in any convenient manner, where two representative means of transformation are treatment with divalent cation transformation compositions and electrotransformation. In transformation through divalent cation treatment, the host cells are typically incubated with the one or more divalent cations, e.g. CaCl₂, which serves to make the host cell permeable to the vector DNA. See Cohen et al. (1972) Proc. Nat'l. Acad. Sci. USA 69:2110. Other agents with which the host cells may also be incubated include DMSO, reducing agents, hexaminecobalt and the like, where such agents serve to improve the efficiency of transformation. In electrotransformation (also known as transformation by electroporation) host cells are subject to an electrical pulse in the presence of the vector in a manner sufficient for the vector to enter the host cells. See Dower et al. (1988) Nucleic Acids Research 16:6127.

A variety of host cells are suitable and may be used in the production of the subject fusion polypeptides. Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142;, Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

Plant cells. Plant cell culture is amply described in various publications, including, e.g., Plant Cell Culture: A Practical Approach, (1995) R. A. Dixon and R. A. Gonzales, eds., IRL Press; and U.S. Pat. No. 6,069,009.

Utility

The subject methods find use in a variety of applications, where detection of the presence of an active protease is of interest. Such applications include, but are not limited to: monitoring activity of a protease in a cell, e.g., to determine whether a particular protease is present or absent in a cell; monitoring the effect of an agent on the activity of a protease, e.g., for drug screening applications to identify agents that modulate the activity of a particular protease; studying the effect of a factor on expression of the protease-encoding gene, e.g., via cotransfection with a second vector encoding the factor of interest; and the like.

As such, one representative application in which the subject methods and compositions find use is in the detection of a protease activity of interest in a cell. Since proteases control various processes in eukaryotic cells, the subject detection applications can be used to determine the particular state of the cell associated with the particular protease. For example, the presence in a cell of particular active caspases indicates that the cell is undergoing an apoptotic event. In addition, protease detection applications can be used in diagnostic applications, including diagnosis of bacterial pathogenic infection, e.g., by detecting the presence of bacterial toxin proteases, the diagnosis of viral pathogenic invention, e.g., by detecting the presence of viral protease activity in a cell; and the like.

Another broad category of applications in which the subject methods and compositions find use is in applications where the effect of a candidate agent is observed on a given protease, e.g., in drug screening applications for the identification of agents that can modulate the activity of a given protease. In such applications, a cell containing a subject fusion protein is useful in drug screening applications to identify agents that modulate the activity and/or expression of a protease. Accordingly, the invention provides methods of identifying an agent that modulates the activity and/or expression of a protease. Such agents are useful to modulate the activity and/or expression of a given protease. For example, agents that increase the activity and/or expression of a protease that is active during apoptosis are useful to induce apoptosis in unwanted cells, e.g., cancerous cells.

The methods of this particular type of application generally involve contacting a cell harboring a subject fusion protein with an agent being tested; and determining the effect, if any, of the agent on the activity and/or expression of the protease. Cells useful in such assays include animal, plant, and yeast cells, including, but not limited to, mammalian cell lines (e.g., 293 cells, COS cells, and the like); insect cell lines (e.g., Drosophila S2 cells, and the like); and plant cell lines.

A variety of different candidate agents (“test agents”) may be screened by the screening methods of the invention. Candidate agents encompass numerous chemical classes, though typically they are organic molecules, and may be small organic compounds having a molecular weight of more than 50 and less than about 2,500 daltons. Candidate agents comprise functional groups necessary for structural interaction with proteins, e.g., hydrogen bonding, and can include at least an amine, carbonyl, hydroxyl or carboxyl group, or at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents, also referred to herein as “test agents,” are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

An “agent that modulates the activity and/or expression of a protease”, as used herein, describes any molecule, e.g. synthetic or natural organic or inorganic compound, protein or pharmaceutical, with the capability of altering the activity of a regulatory element, as described herein. Generally a plurality of assay mixtures is run in parallel with different agent concentrations to obtain a differential response to the various concentrations. Typically, one of these concentrations serves as a negative control, i.e. at zero concentration or below the level of detection. The activity of the protease is determined by determining the subcellular location of the fluorescent protein, as described above.

The subject methods and compositions are amenable to use in high throughput applications. For example, where one is interested in the high throughput screening of the effect of a library of agents on the activity of a protease, a plurality of test cells can be provided, e.g., in a multiwell plate, and each test cell exposed to a different agent of the library, where each agent is then monitored for its effect on the protease activity of interest in the cell. Other high throughput formats are also amenable, e.g., flow cytometry formats, high throughput cell based screening protocols, e.g., as described in U.S. Pat. Nos. 5,989,835; 6,103,479; and 6,365,367; the disclosures of which are herein incorporated by reference.

Systems

Also provided are systems for use in practicing the subject methods. The subject systems at least include a protease detection fusion protein or nucleic acid coding sequence therefore, e.g., present on a suitable vector, as described above. In addition, the subject systems include a cell to be assayed. In certain embodiments, the two components are combined, e.g., the vector is present in the cell to be assayed. In yet other embodiments, the two components are not yet combined, e.g., where the system is not yet being employed. Other components of the subject systems include, but are not limited to: reaction buffer, controls, etc.

Kits

Also provided by the subject invention are kits for use in practicing the subject methods, where the subject kits and/or systems include at least a fusion protein according to the subject invention, or a nucleic acid, e.g., present in a construct, comprising a nucleotide sequence that includes a coding region for a fusion protein, as described above. The above components may be present in a suitable storage medium, e.g., buffered solution, typically in a suitable container.

In certain embodiments, the kit comprises a plurality of different vectors each encoding a subject fusion protein, where the vectors are designed for expression in different environments and/or under different conditions, e.g., a vector which includes a cloning site for insertion of a DNA fragment encoding a protease cleavage site; a number of vectors, each of which includes a coding sequence for a different protease cleavage site, etc.

More than one restriction endonuclease site may be provided in a tandem and/or partially overlapping arrangement, such that a “multiple cloning site” is provided. The recombinant vector may further comprise control sequences, such as a promoter, a translation initiation site, a polyadenylation site, and the like, for controlling expression of the coding region in prokaryotic or eukaryotic cells. The kit may further comprise appropriate restriction enzyme(s), ligases, and other reagents for inserting a heterologous nucleic acid molecule into the recombinant vector.

The kit may further include a double-stranded nucleic acid molecule with 5′ and/or 3′ overhanging ends, which double-stranded nucleic acid molecule includes a nucleotide sequence encoding a protease cleavage site, and, on the 5′ and 3′ ends of the double-stranded nucleic acid molecule, overhanging ends that are complementary to overhanging ends of a recombinant construct as described above, linearized with an appropriate restriction endonuclease. The double-stranded nucleic acid molecule can be ligated to a linearized recombinant construct such that the construct encodes a fusion protein as described above.

The kit may further comprise bacteria for propagating the recombinant vector; reagents for introducing the recombinant vector into the bacteria; and reagents for selecting bacteria that comprise the recombinant vector.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE I

### Caspase3 Assay Using an NES-Protease Cleavage-NLS Fusion Protein

A construct was generated that includes a nucleotide sequence encoding a fusion protein including, in order from amino to carboxyl terminus, an NES of MAP-kinase-kinase (NLVDLQKKLEELELDEQQ; SEQ ID NO: 23); a recognition site for caspase-3 (DEVD; SEQ ID NO: 22) bordered by a stretch of amino acids found in the cleavage site of the endogenous caspase-3 substrate poly (ADP-ribose) polymerase (PARP; Nicholson et al. (1995) Nature 376:37-43; and Tewari et al. (1995) Cell 81:801-809), such that the cleavage recognition site has the sequence KRKGDEVDGVDF (SEQ ID NO: 24); an enhanced yellow fluorescent protein (EYFP); and a three tandem repeat of the NLS from simian virus large T antigen. In this construct, the NES is dominant over the NLS.

The construct was transfected into mammalian cells. Specifically, 3T3 cells were grown on coverslips, transiently transfected with the pCaspase3-sensor Vector which is encodes the above described fusion protein and is further illustrated in Clontechniques (April, 2002), and grown for 24 hours. Apoptosis was induced using staurosporin (700 nM) and caspase-3 activity was detected 4 hours post induction. Cells were fixed with 3% paraformaldehyde and photomicrographs were taken using a Zeiss microscope.

It was observed that when the caspase-3 was inactive, e.g., in the cells not treated with staursporin, the EYFP localized in the cytoplasm. However, when caspase-3 was active due to induction with staurosporin, the NES was cleaved from the fusion protein, and the EYFP was detected in the nucleus.

The above assay is further illustrated in FIG. 1.

### EXAMPLE II

### Caspase3 Assay Using an Plasma Membrane Localization Domain-Protease Cleavage-NES Fusion Protein

An additional way to use a translocation event as a “readout” to monitor cytosolic protease activity is to construct a fusion protein that contains, instead of a dominant NES as described above, a domain that contains the signal sequence for a posttranslational myristylation or farnesylation event. In this case the uncleaved fusion protein containing the myristylated or farnesylated domain, a protease cleavage site, a label domain and a NLS, would associate with the inner (cytosolic) leaflet of the plasmamembrane. Upon activation of the protease of interest, the protein would be cleaved, releasing the label domain containing the NLS from the plasmamembrane localization, allowing it to transfer into the nucleus, driven by the NLS. This assay is further illustrated in FIG. 2.

It is apparent from the above discussion that the invention provides methods for detecting the presence of an active protease in a cell, using translocation of a fluorescent protein as the readout. Such methods are useful in various applications, e.g., monitoring the activity of a protease, drug screening applications, and the like. Because one need not lyse a cell in order to obtain information about an active protease therein, and may practice the methods in vivo and in real time, the subject invention provides for a number of distinct advantages over that which is available by the prior art protocols described in the Background section, above. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of determining whether a cell contains an active protease in a cell, said method comprising:
(a) providing a protease detection fusion protein in said cell, wherein said protease detection fusion protein comprises first and second subcellular localization domains separated from each other by a cleavage domain recognized by said protease, and further wherein said first subcellular localization domain is dominant over said second subcellular localization domain and a label domain is present between said cleavage and second subcellular localization domains;
(b) maintaining said cell for a period of time sufficient for said protease cleavage domain to be cleaved by said protease if present in said cell; and
(c) detecting the subcellular location of said label domain to determine whether said cell contains said active protease.

2. The method according to claim 1, wherein said protease activity is detected in, a single, live cell.

3. The method according to claim 1, wherein said protease activity is detected in real time.

4. The method according to claim 1, wherein said label domain is a fluorescent protein.

5. The method according to claim 1, wherein said first localization domain is a nuclear export signal (NES).

6. The method according to claim 1, wherein said second localization domain is a nuclear localization signal (NLS).

7. A fusion protein comprising:
(a) a first subcellular localization domain;
(b) a protease cleavage domain;
(c) a label domain; and
(d) a second subcellular localization domain,
wherein the first localization domain is dominant over the second localization domain.

8. The fusion protein according to claim 7, wherein said label domain is a fluorescent protein.

9. The fusion protein according to claim 7, wherein said first localization domain is a nuclear export signal (NES).

10. The fusion protein according to claim 7, wherein said second localization domain is a nuclear localization signal (NLS).

11. The fusion protein according to claim 7, wherein said protease cleavage site is D-E-V-D (SEQ ID NO: 22)

12. A nucleic acid encoding a fusion protein according to claim 7.

13. A vector comprising a nucleic acid according to claim 12.

14. The vector according to claim 13, wherein said vector is an expression vector.

15. A cell that includes a nucleic acid according to claim 12.

16. The cell according to claim 15, wherein said cell is a eukaryotic cell.

17. A system for detecting the presence of an active protease in a cell, the system comprising:
(a) a fusion protein according to claim 7 or a nucleic acid encoding the same; and
(b) said cell.

18. A kit for use in detecting the presence of an active protease in a cell, said kit comprising:
(a) a fusion protein according to claim 7 or a nucleic acid encoding the same; and
(b) instructions for practicing a method according to claim 1.

19. The kit according to claim 18, wherein said kit further comprises a cell.
